(19) United States
(12) Patent Application Publication
(10) Pub. No.: US 2025/0025461 A1
(43) Pub. Date: Jan. 23, 2025

(54) TREATMENT OF SEVERE AND UNCOMPLICATED MALARIA

(71) Applicants: PURDUE RESEARCH FOUDATION, West Lafayette, IN (US); UNIVERSITY OF SASSARI, Sassari (IT); NUREX S.R.L., Sassari (IT); VINUNIVERSITY, Hanoi (VN)

(72) Inventors: Philip Stewart LOW, West Lafayette, IN (US); Huynh Dinh Chien, Hanoi (VN); Francesco Michelangelo Turrini, Sassari (IT); Antonella Pantaleo, Sassari (IT)

(21) Appl. No.: 18/291,413

(22) PCT Filed: Jul. 22, 2022
(86) PCT No.: PCT/US2022/074071

§ 371 Date: Jan. 23, 2024

(30) Foreign Application Priority Data
Jul. 23, 2021 (VN) 1-2021-04540

(51) Int. Cl. A61K 31/506; A61K 9/20; A61K 31/357; A61K 31/496; A61P 33/06

(52) U.S. Cl. CPC A61K 31/506; A61K 9/20; A61K 31/357; A61K 31/496; A61P 33/06

(57) ABSTRACT

Methods of treating severe and uncomplicated malaria comprising administering an isoform of artemisinin, a hydrophobic amine, and a spleen tyrosine kinase (Syk) inhibitor. Further provided is a unitary, oral dosage form comprising an isoform of artemisinin, a hydrophobic amine, and a Syk inhibitor; and a kit comprising multiple unitary, oral dosage forms and instructions for administration.

### STATEMENT OF GOVERNMENT SUPPORT

The invention was made with government support under grant no. GM24417-40 awarded by the National Institutes of Health. Therefore, the government of the United States of America has certain rights in the claimed invention.

### CROSS REFERENCE TO RELATED APPLICATIONS

This application claims priority to Vietnamese Application No. 1-2021-04540, which was filed on Jul. 23, 2021, and which is hereby incorporated by reference in its entirety.

### TECHNICAL FIELD

The present disclosure relates to methods of treating severe and uncomplicated malaria; isoforms of artemisinin; hydrophobic amines; spleen tyrosine kinase (Syk) inhibitors; unitary, oral dosage forms; and kits.

### BACKGROUND

Malaria remains a serious health problem in much of the world today, with 228 million new cases and 405,000 deaths reported in 2018 (World Health Organization. World Malaria Report 2019. Geneva: World Health Organization; 2019. Pages 1-232). Although artemisinin combination therapies (ACTs) continue to treat successfully most strains of P. falciparum malaria, new drug-resistance mutations leading to delayed parasite clearance (DPC; continued parasitemia following 3 days of standard therapy) are now emerging (Conrad et al., Lancet Infect Dis 19 (10): e-338-351 (October 2019); Ouji et al., Parasite [Internet] 25:24 (Apr. 20, 2018); Lu et al., N Engl J Med [Internet] 376 (10): 991-993 (2017); Pau et al., PLOS One 14 (4): e0214667 (2019); and Thriemer et al., Antimicrob Agents Chemother [Internet] 58 (12): 7049 LP-7055 (Dec. 1, 2014)) suggesting that current measures to control parasite propagation may soon be inadequate. While artemisinin, the cornerstone of ACTs, is fast-acting and effective, the duration of its efficacy is short, requiring a companion drug to achieve more prolonged activity (Nsanzabana, Top Med Infect Dis [Internet] 4 (1): 26 (February 2019); Li, Chapter 4, Pharmacokinetic and Pharmacodynamic Profiles of Rapid- and Slow-Acting Antimalarial Drugs. In: Kasenga BPE-FH, editor. Rijeka: IntechOpen (2019)). Unfortunately, resistance to such companion drugs is also rising (Conrad et al. (2019), supra), and while triple combination therapies are now under investigation for prevention of DPC (Rosenthal, Lancet [Internet] 395 (10233): 1316-1317 (Apr. 25, 2020); van der Pluijm et al., Lancet [Internet] 395 (10233): 1345-1360 (Apr. 25, 2020); and Dini et al., Antimicrob Agents Chemother [Internet] 62 (11): e01068-18 (Nov. 1, 2018)), the third components in most such ACTs are anti-malarials that have already failed due to decline in efficacy (Conrad et al. (2019), supra). Taken together, these observations suggest that new approaches to treat P. falciparum malaria with orthogonal mechanisms of action are critically needed.

In view of the above, it is an object of the present disclosure to provide a triple combination therapy for the treatment of severe and uncomplicated malaria. This and other objects and advantages, as well as inventive features, will be apparent from the detailed description provided herein.

### SUMMARY

A method of treating severe malaria in a subject is provided. The method comprises administering to the subject an isoform of artemisinin, a hydrophobic amine, and a spleen tyrosine kinase (Syk) inhibitor in amounts effective to eliminate parasitemia within about 72 hours.

In the method of treating severe malaria, the isoform of artemisinin can be selected from the group consisting of artemisinin, dihydroartemisinin, artesunate, and artemether. The hydrophobic amine in the method of treating severe malaria can be selected from the group consisting of lumefantrine, mefloquine, amodiaquine, the combination of sulfadoxine and pyrimethamine, piperaquine, chloroquine, and the combination of chlorproguanil and dapsone.

The Syk inhibitor in the method of treating severe malaria can, and preferably does, compete with adenosine triphosphate (ATP) for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be selected from the group consisting of imatinib, imatinib mesylate, and nilotinib. The Syk inhibitor can be Syk inhibitor II, Syk inhibitor IV, R406 (active metabolite of fostamatinib), R788 (fotamatinib), P505-15 (PRT062607), MNS (3,4-methylenedioxy-β-nitrostyrene), R112, GS-9973 (entospletinib), piceatannol, dasatinib, bosutinib, or ponatinib.

The method of treating severe malaria can comprise administering to the subject about 40 mg/day dihydroartemisinin, about 320 mg/day piperaquine, and about 400 mg/day imatinib.

A method of treating uncomplicated malaria in a subject is also provided. The method comprises administering to the subject an isoform of artemisinin, a hydrophobic amine, and a Syk inhibitor in amounts effective to eliminate parasitemia within about 72 hours, wherein, when the isoform of artemisinin is dihydroartemisinin and the hydrophobic amine is piperaquine, the Syk inhibitor is other than imatinib or imatinib mesylate.

In the method of treating uncomplicated malaria, the isoform of artemisinin can be selected from the group consisting of artemisinin, dihydroartemisinin, artesunate, and artemether. The hydrophobic amine in the method of treating uncomplicated malaria can be selected from the group consisting of lumefantrine, mefloquine, amodiaquine, the combination of sulfadoxine and pyrimethamine, piperaquine, chloroquine, and the combination of chlorproguanil and dapsone.

The Syk inhibitor in the method of treating uncomplicated malaria can, and preferably does, compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be nilotinib. The Syk inhibitor can be Syk inhibitor II, Syk inhibitor IV, R406 (tamatinib; active metabolite of fostamatinib), R788 (fotamatinib), P505-15 (PRT062607), MNS (3,4-methylenedioxy-β-nitrostyrene), R112, GS-9973 (entospletinib), piceatannol, dasatinib, bosutinib, or ponatinib.

Another method of treating uncomplicated malaria is also provided. The method comprises administering to the subject about 40 mg/day dihydroartemisinin, about 320 mg/day piperaquine, and about 400 mg/day imatinib.

Further provided is a unitary, oral dosage form comprising an isoform of artemisinin, a hydrophobic amine, and a Syk inhibitor in amounts effective to treat parasitemia. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can, and preferably does, compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be selected from the group consisting of imatinib, imatinib mesylate, and nilotinib. The Syk inhibitor can be Syk inhibitor II, Syk inhibitor IV, R406, R788, P505-15, 3,4-methylenedioxy-β-nitrostyrene, R112, GS-9973, piceatannol, dasatinib, bosutinib, or ponatinib. The unitary, oral dosage form can comprise dihydroartemisinin, piperaquine, and imatinib, such as about 40 mg dihydroartemisinin, about 320 mg piperaquine, and about 400 mg imatinib.

The unitary, oral dosage form can comprise an isoform of artemisinin, a hydrophobic amine, and a Syk inhibitor other than imatinib or imatinib mesylate. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can, and preferably does, compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be nilotinib. The Syk inhibitor can be Syk inhibitor II, Syk inhibitor IV, R406, R788, P505-15, 3,4-methylenedioxy-β-nitrostyrene, R112, GS-9973, piceatannol, dasatinib, bosutinib, or ponatinib. The unitary, oral dosage form can comprise dihydroartemisinin, piperaquine, and a Syk inhibitor other than imatinib or imatinib mesylate, such as about 40 mg dihydroartemisinin, about 320 mg piperaquine, and about 400 mg of a Syk inhibitor other than imatinib or imatinib mesylate.

Still further provided is a kit. The kit comprises multiple unitary, oral dosage forms as described above and instructions for administration for severe malaria, uncomplicated malaria, or both. The kit can comprise two or three unitary, oral dosage forms.

### BRIEF DESCRIPTION OF THE FIGURES

FIG. 1A is a graph of days vs. average parasitemia (parasites/μL) for standard of care (SOC; 40 mg dihydroartemisinin+320 mg piperaquine phosphate; n=8) and imatinib only (400 mg; n=7). Error bars are expressed as SEM.

FIG. 1B is a graph of days vs. parasitemia (parasites/μL), which shows the representative time course of parasitemia in a participant in the imatinib monotherapy trial who exhibited a temporary rise in parasitemia.

FIG. 1C is a graph of days vs. parasitemia (parasites/μL), which shows the representative time course of parasitemia in a participant in the imatinib monotherapy trial who exhibited a monotonous decrease in parasitemia.

FIG. 1D is a graph of days vs. temperature (° C.), which shows the average participant body temperatures plotted for SOC and imatinib only cohorts. Error bars are expressed as SEM.

FIG. 2A is a graph of days vs. temperature (° C.), which shows the daily participant body temperatures plotted for SOC (40 mg dihydroartemisinin+320 mg piperaquine phosphate; n=21) and SOC+imatinib (SOC+400 mg imatinib; n=20). Error bars expressed as SEM and *=p-value <0.05, **=p-value <0.01, ***=p-value <0.001, and ****=p-value <0.0001.

FIG. 2B is a bar graph of SOC and SOC+imatinib (Im+SOC) vs. pyrexia duration (days), which shows the average duration of pyrexia in the two cohorts. Error bars expressed as SEM and *=p-value <0.05, **=p-value <0.01, ***=p-value <0.001, and ****=p-value <0.0001.

FIG. 2C is a bar graph of SOC and Im+SOC vs. % of patients, which shows the percentage of participants that exhibited a second fever spike. Error bars expressed as SEM and *=p-value <0.05, **=p-value <0.01, ***=p-value <0.001, and ****=p-value <0.0001.

FIG. 2D is a graph of days vs. temperature (° C.), which shows the plots of individual body temperatures over time for the SOC cohort (n=21).

FIG. 2E is a graph of days vs. temperature (° C.), which shows the plots of individual body temperatures over time for the Im+SOC cohort (n=20).

FIG. 3A is a graph of days vs. average parasitemia (parasites/u L) for SOC and Im+SOC. Error bars expressed as SEM and *=p-value <0.05, **=p-value <0.01, ***=p-value <0.001, and ****=p-value <0.0001.

FIG. 3B is a graph of days vs. patients with detectable parasitemia (%), which shows the percentage of patient with detectable parasitemia on different days post-initiation of therapy. Error bars expressed as SEM and *=p-value <0.05, **=p-value <0.01, ***=p-value <0.001, and =p-value <0.0001.

FIG. 3C is a bar graph of SOC and Im+SOC vs. time to reach 50% of starting parasitemia (h), which shows the average time for parasitemia to decline to 50% of its starting level. Error bars expressed as SEM and *=p-value <0.05, **=p-value <0.01, ***=p-value <0.001, and ****=p-value <0.0001.

FIG. 3D is a graph of days for SOC (starting parasitemia <10,000 parasites/μL and >10,000 parasites/μL) and Im+SOC (starting parasitemia <10,000 parasites/u L and >10,000 parasites/μL) vs. parasitemia remaining (%), which shows the decrease in parasitemia as a function of time. Error bars expressed as SEM and *=p-value <0.05, **=p-value <0.01, ***=p-value <0.001, and ****=p-value <0.0001.

FIG. 3E is a graph of days vs. % parasitemia remaining for SOC (starting parasitemia <10,000; n=9).

FIG. 3F is a graph of days vs. % parasitemia remaining for Im+SOC (starting parasitemia <10,000; n=10).

FIG. 3G is a graph of days vs. % parasitemia remaining for SOC (starting parasitemia >10,000; n=12).

FIG. 3H is a graph of days vs. % parasitemia remaining for Im+SOC (starting parasitemia >10,000; n=10).

### DETAILED DESCRIPTION

Malaria in humans is caused by five species of single-celled, eukaryotic parasites known as Plasmodium. The main species, which cause malaria, are P. falciparum and P. vivax. The parasites are transmitted to humans by mosquito bites, in particular bites by Anopheles mosquitoes.

The parasites grow and multiply in the liver. Then they grow exponentially in red blood cells. It is at this stage of the life cycle of the parasite that signs and symptoms of infection are apparent.

Malaria is typically classified as asymptomatic, uncomplicated, and complicated/severe. A patient suffering from “asymptomatic malaria” has circulating parasites but no symptoms. “Uncomplicated malaria” usually presents within 7-10 days after a bite by an infectious mosquito. Symptoms are non-specific and can include fever, chills, shaking, profuse sweating, headache, nausea, vomiting, diarrhea, and anemia. “Complicated/severe malaria” is usually caused by infection with P. falciparum, although it can be caused by infection with P. vivax or P. knowlesi. It is accompanied by severe anemia and clinical and laboratory signs of severe organ dysfunction, including cerebral malaria (e.g., abnormal behavior, impaired consciousness, seizures, coma, and other neurologic abnormalities), pulmonary complications (e.g., edema and hyperpneic syndrome), hypoglycemia, and acute kidney injury. It is often associated with hyperparasitemia (e.g., >5% infected erythrocytes or >250,000 parasites/μl); and death often results (e.g., 3% with >4% infected erythrocytes).

In view of the foregoing, a method of treating severe malaria in a subject is provided. The method comprises administering to the subject an isoform of artemisinin, a hydrophobic amine, and a spleen tyrosine kinase (Syk) inhibitor in amounts effective to eliminate parasitemia within about 72 hours. Parasitemia can be eliminated within about 48 hours. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can compete with adenosine triphosphate (ATP) for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be selected from the group consisting of imatinib, imatinib mesylate, and nilotinib. The Syk inhibitor can be Syk inhibitor II, Syk inhibitor IV, R406 (tamatinib; active metabolite of fostamatinib), R788 (fotamatinib), P505-15 (PRT062607), MNS (3,4-methylenedioxy-β-nitrostyrene), R112, GS-9973 (entospletinib), piceatannol, dasatinib, bosutinib, or ponatinib. There can be, and desirably is, synergism between (i) the combination of the isoform of artemisinin and the hydrophobic amine and (ii) the Syk inhibitor. The method can comprise administering to the subject about 40 mg/day dihydroartemisinin, about 320 mg/day piperaquine, and about 400 mg/day imatinib.

Also in view of the above, a method of treating uncomplicated malaria in a subject, which method comprises administering to the subject an isoform of artemisinin, a hydrophobic amine, and a Syk inhibitor in amounts effective to eliminate parasitemia within about 72 hours (e.g., within about 48 hours), wherein, when the isoform of artemisinin is dihydroartemisinin and the hydrophobic amine is piperaquine, the Syk inhibitor is other than imatinib or imatinib mesylate, whereupon the subject is treated for uncomplicated malaria. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be nilotinib. The Syk inhibitor can be Syk inhibitor II, Syk inhibitor IV, R406 (tamatinib; active metabolite of fostamatinib), R788 (fotamatinib), P505-15 (PRT062607), MNS (3,4-methylenedioxy-β-nitrostyrene), R112, GS-9973 (entospletinib), piceatannol, dasatinib, bosutinib, or ponatinib.

Another method of treating uncomplicated malaria is also provided. The method comprises administering to the subject about 40 mg/day dihydroartemisinin, about 320 mg/day piperaquine, and about 400 mg/day imatinib.

“Administering” can be by any suitable route as known in the art. The administration can be oral, such as oral administration of a unitary, oral dosage form as described below.

The isoform of artemisinin, the hydrophobic amine, and the Syk inhibitor (i.e., “active agents”), or compositions comprising same, can be administered by the same or different routes and/or at the same or different times. Desirably, however, the active agents are administered by such routes and at such times as to allow, and even promote, synergy between the active agents.

The active agents are administered in amounts effective to treat parasitemia. Synergy between the active agents enables lower daily dosages to be administered.

The isoform of artemisinin can be administered in any suitable daily amount, such as an amount less than about 100 mg, such as about 95 mg, about 90 mg, about 85 mg, about 80 mg, about 75 mg, about 70 mg, about 65 mg, about 60 mg, about 55 mg, about 50 mg, about 45 mg, about 40 mg, about 35 mg, about 30 mg, or about 25 mg. The isoform of artemisinin can be administered daily in an amount of about 40 mg.

The hydrophobic amine can be administered in any suitable daily amount, such as an amount less than about 1,000 mg, such as about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, or about 250 mg. The hydrophobic amine can be administered daily in an amount of about 320 mg.

The Syk inhibitor can be administered in any suitable daily about, such as an amount less than about 1,000 mg, such as about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, or about 250 mg. The Syk inhibitor can be administered daily in an amount less than about 750 mg, about 700 mg, about 650 mg, about 600 mg, about 550 mg, about 500 mg, about 450 mg, about 400 mg, about 350 mg, about 300 mg, or about 250 mg. The Syk inhibitor can be administered daily in an amount of about 40 mg.

Further provided is a unitary, oral dosage form. The unitary, oral dosage form can comprise an isoform of artemisinin, a hydrophobic amine, and a Syk inhibitor in amounts effective to treat parasitemia. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can, and preferably does, compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be selected from the group consisting of imatinib, imatinib mesylate, and nilotinib. The Syk inhibitor can be Syk inhibitor II, Syk inhibitor IV, R406, R788, P505-15, 3,4-methylenedioxy-β-nitrostyrene, R112, GS-9973, piceatannol, dasatinib, bosutinib, or ponatinib. The unitary, oral dosage form can comprise dihydroartemisinin, piperaquine, and imatinib, such as about 40 mg dihydroartemisinin, about 320 mg piperaquine, and about 400 mg imatinib.

The unitary, oral dosage form can comprise an isoform of artemisinin, a hydrophobic amine, and a Syk inhibitor other than imatinib or imatinib mesylate. The isoform of artemisinin can be selected from the group consisting of artemisinin, dihydroartemisinin, artesunate, and artemether. The hydrophobic amine can be selected from the group consisting of lumefantrine, mefloquine, amodiaquine, the combination of sulfadoxine and pyrimethamine, piperaquine, chloroquine, and the combination of chlorproguanil and dapsone. The Syk inhibitor can, and preferably does, compete with ATP for binding to Syk. The Syk inhibitor can comprise a bisarylanilino core that interacts with the gatekeeper amino acid residue Thr315 of the ATP binding pocket of BCR-ABL through hydrogen bond and Van der Waals interactions. The Syk inhibitor can be nilotinib. The Syk inhibitor can be Syk inhibitor II, Syk inhibitor IV, R406, R788, P505-15, 3,4-methylenedioxy-β-nitrostyrene, R112, GS-9973, piceatannol, dasatinib, bosutinib, or ponatinib. The unitary, oral dosage form can comprise dihydroartemisinin, piperaquine, and a Syk inhibitor other than imatinib or imatinib mesylate, such as about 40 mg dihydroartemisinin, about 320 mg piperaquine, and about 400 mg of a Syk inhibitor other than imatinib or imatinib mesylate.

The unitary, oral dosage form can comprise a pharmaceutically acceptable carrier or excipient as known in the art. A pharmaceutically acceptable carrier or excipient is useful in preparing a pharmaceutical composition that is generally safe, non-toxic and neither biologically nor otherwise undesirable. Such carriers and excipients can include those that are acceptable for human pharmaceutical use and veterinary use.

Still further provided is a kit. The kit comprises multiple, such as at least two or three, e.g., two or three, unitary, oral dosage forms and instructions for administration for severe malaria or uncomplicated malaria (or both).

### Example

The following example serves to illustrate aspects of the present disclosure. The example is not intended to limit the scope of the claimed invention.

### Study Agents

Generic imatinib mesylate was purchased from TEVA Pharmaceuticals (Jerusalem, Israel) and was provided to patients in two 200 mg tablets per dose to conform with the usual quantity of imatinib indicated for treatment of chronic myelogenous leukemia. CV-Artecan, the standard-of-care (SOC) for treatment of P. falciparum malaria in Vietnam was purchased from OPC Pharmaceutical (Ho Chi Minh City, Vietnam) and was administered to patients as a tablet containing 40 mg dihydroartemisinin plus 320 mg piperaquine phosphate.

### Study Participants

P. falciparum-infected males of age 16-55 years with no complicating co-morbidities that had not received an anti-malarial drug within the previous 4 weeks were eligible for two trials, i.e., imatinib monotherapy and imatinib plus SOC (Im+SOC) triple combination therapy. Those individuals who met eligibility criteria and provided informed written consent were enrolled in the trial.

### Study Protocols

The phase 1/2 “imatinib monotherapy trial” was an open-label trial aimed at determining the safety and tolerability of imatinib in adult male patients with uncomplicated P. falciparum malaria. Patients were randomly assigned to either a standard-of-care (SOC) control arm (n=8) in which they received 40 mg dihydroartemisinin plus 320 mg piperaquine phosphate orally twice (12 hours apart) on the first day and then once a day on the following 2 days, or an imatinib monotherapy arm (n=7) in which they received 400 mg of imatinib mesylate orally with a meal and full glass of water once a day for five days.

Participants in the subsequent “imatinib+SOC (Im+SOC) triple combination therapy trial” were also randomly assigned to one of two cohorts, either the SOC cohort (n=21) or an Im+SOC cohort (n=20). The SOC cohort was dosed exactly as above, while the Im+SOC cohort was dosed as described above except each patient also received 400 mg of imatinib mesylate orally with a meal and full glass of water once a day for 3 days.

During both trials, patient temperatures and peripheral blood parasite levels were monitored before, during and after the trial on days 0, 1, 2, 3, 5, 7, 28, and 42. Patients were also examined for the usual symptoms of P. falciparum malaria, including fever, chills, headache, fatigue, anorexia, and mild diarrhea. If any participant was observed to exhibit either an increase in parasitemia >150,000 parasites/μL or adverse symptoms exceeding those normally associated with malaria, the patient was transferred immediately to SOC.

Both trials were approved by the Vietnam Ministry of Health and the Institutional Review Board at the Hue University of Medicine and Pharmacy. The trials were conducted in the six Communes of the Lia Region Huong Hoa district, Quang Tri province, because this region of Vietnam has high levels of DPC (Pau et al. (2019), supra; and Thriemer et al. (2014), supra). Both studies were registered online with ClinicalTrials(dot)gov (NCT02614404 and NCT03697668).

### Randomization and Masking

Both clinical trials were open label, so no blinding of the attending physicians was performed. Participants, however, were randomly assigned to their cohorts by alternating their assignment based on the date and time of hospital admission, and all participants as well as microscopists who quantitated the parasitemia were blinded to the treatment regimens.

### Outcomes

Primary endpoints for both studies were safety and tolerability. Secondary endpoints were reduction in parasitemia for the imatinib monotherapy study and both reduction in parasitemia and decline in pyrexia for the Im+SOC study. Safety and tolerability were assessed at protocol-specified time points and adverse events were classified as toxicities not normally associated with P. falciparum malaria, including edema, rashes, and severe diarrhea, etc. The severity of any adverse event was proposed to be classified as follows: i) Mild-events requiring minimal or no treatment that did not interfere with the participant's daily activities, ii) Moderate-events resulting in a low level of inconvenience or concern that may have caused some interference with the participant's daily functioning, and iii) Severe-events that interrupt a participant's daily activity and could be incapacitating or require medical intervention. Attribution of imatinib to adverse events was assessed using a 5-point scale: not related, unlikely related, possibly related, probably related, and definitely related. Primary endpoints would be met if the imatinib treatment groups exhibited an absence of any severe adverse events and an insignificant increase or actual decrease in moderate adverse events.

### Statistical Analysis

A mixed ANOVA was conducted to determine the effect of drug treatment on parasitemia and pyrexia. Post-hoc multi-comparison testing was used to determine which time points were statistically different. Two-tail t-tests were used to determine statistical differences between means of independent groups. Significance was assumed for p-values <0.05. Data from all participants who received three doses of treatment drug(s) were included in the analysis. SOC treatment cohorts from each individual trial were analyzed separately. Unless stated otherwise, all error bars represent standard error of the mean (SEM).

### Results

To assess the safety, tolerability, and efficacy of imatinib in patients with P. falciparum malaria, an initial Phase 1/2 clinical trial was conducted where imatinib was administered to participants as a monotherapy and compared with a parallel cohort treated with standard-of-care (SOC) therapy in Vietnam. Although imatinib had already established a good safety record when administered in perpetuity to chronic myelogenous leukemia cancer patients (O'Brien et al., N Engl J Med 348 (11): 994-1004 (2003); and Hochhaus et al., N Engl J Med [Internet] 376 (10): 917-927 (Mar. 9, 2017)), it had not been dosed in malaria patients prior to this study. Therefore, the primary endpoint for this trial was the safety and tolerability of imatinib in patients with P. falciparum malaria and the secondary endpoint was reduction in parasitemia.

Before the trial could begin, an appropriate site with endemic malaria and delayed parasite clearance (DPC) needed to be identified. As noted in the introduction, DPC had been shown to be increasing in Southeast Asia, requiring treatment of patients well beyond the usual three days of therapy (Pau et al. (2019), supra; and Thriemer et al. (2014), supra). Because any new remedy for malaria would have to demonstrate efficacy against these more refractory strains of P. falciparum malaria, it was decided to conduct the initial clinical trial in the Quang Tri Province of Vietnam, where DPC had been documented by standard microscopy in 27.2% of the patients (39.3% when parasitemia was measured by PCR) and genetic markers of artemisinin and piperaquine resistance (K13 C580Y and PfPM2 multi-copies) had been identified in 1.2% of infected individuals (Pau et al. (2019), supra).

To evaluate imatinib's safety and tolerability, participants (males, 18 to 54 years of age; Table 1) with uncomplicated malaria and no comorbidities who had not taken an anti-malaria drug during the preceding month were randomized into one of two treatment cohorts. Following informed consent, participants were treated with either SOC for three days or a single daily dose of 400 mg imatinib (i.e., the usual dose for treatment of chronic myelogenous leukemia patients) for five consecutive days, as described in Methods and Table 2. During and after the therapy, each patient was monitored for changes in hematology, blood chemistry, pyrexia, parasite level, and adverse events. Other than the expected symptoms of P. falciparum malaria, no other adverse events were observed except one case of mild abdominal pain that resolved spontaneously and is periodically observed in malaria patients receiving SOC. Analyses of blood parameters and body temperatures also showed no evidence of drug-related toxicity. As the imatinib monotherapy appeared safe and well tolerated, this trial met its primary endpoint and provided motivation to test imatinib in combination with SOC.

TABLE 1
Baseline characteristics of participants

| | Imatinib only study cohorts | | Imatinib + SOC study cohorts | |
|---|---|---|---|---|
| | SOC | Imatinib only | SOC | Im + SOC |
| | n = 8 | n = 7 | n = 21 | n = 17 |
| Age, years | 20.3 (6.0; | 35.3 (12.7; | 29.1 (6.7; | 25.1 (7.1; |
| (SD; range) | 18-35) | 18-54) | 18-38) | 16-39) |
| Sex | | | | |
| Female | 0 | 0 | 0 | 0 |
| Male | 8 (100%) | 7 (100%) | 21 (100%) | 17 (100%) |
| Race | | | | |
| Southeast Asian | 8 (100%) | 7 (100%) | 21 (100%) | 17 (100%) |

Data are mean (SD) or n(%) unless otherwise stated

TABLE 2
Treatment schedules for trials

| | Cohorts | 0 hours | | 12 hours | | 24 hours | | 48 hours | | 72 hours | 96 hours |
|---|---|---|---|---|---|---|---|---|---|---|---|
| Imatinib | SOC | 40 | mg DHA | 40 | mg DHA | 40 | mg DHA | 40 | mg DHA | NA | NA |
| (Im) only | | 320 | mg PPQ | 320 | mg PPQ | 320 | mg PPQ | 320 | mg PPQ | | |
| Trial | Im | 400 | mg | NA | | 400 | mg | 400 | mg | 400 mg | 400 mg |
| Im + SOC | SOC | 40 | mg DHA | 40 | mg DHA | 40 | mg DHA | 40 | mg DHA | NA | NA |
| Trial | | 320 | mg PPQ | 320 | mg PPQ | 320 | mg PPQ | 320 | mg PPQ | | |
| | Im + SOC | 40 | mg DHA | 40 | mg DHA | 40 | mg DHA | 40 | mg DHA | NA | NA |
| | | 320 | mg PPQ | 320 | mg PPQ | 320 | mg PPQ | 320 | mg PPQ | | |
| | | 400 | mg Im | 400 | mg Im | 400 | mg Im | 400 | mg Im | | |

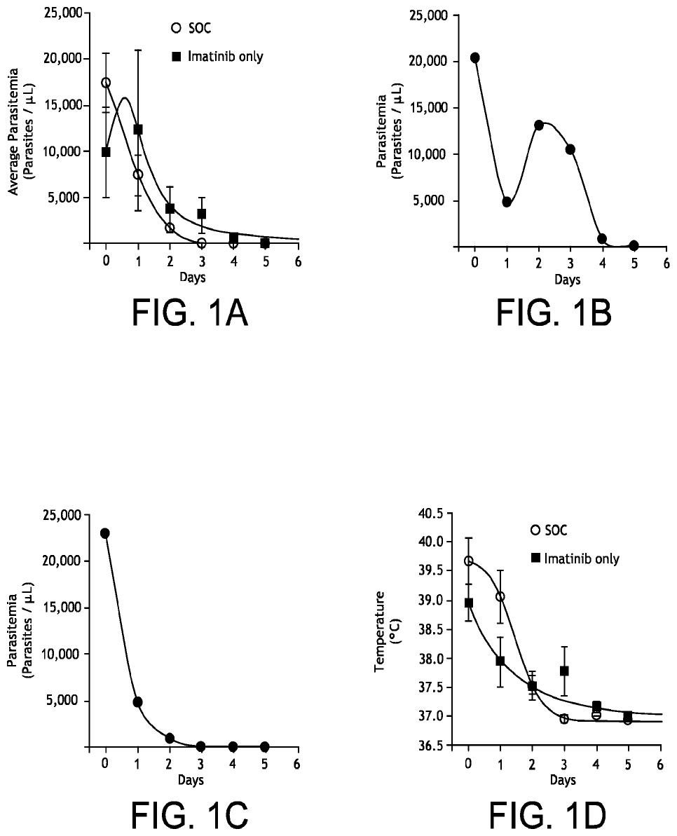

To determine whether imatinib alone might exhibit some hint of efficacy, we concurrently quantitated the parasitemia in the peripheral blood of each participant before, during and after their course of treatment. As seen in FIG. 1A, a decrease in the number of parasitized cells per microliter of blood was observed in the imatinib-treated group. Although this decrease lagged behind the analogous response in the SOC group, when a main factors analysis using a mixed ANOVA was conducted, no statistically significant interaction was identified between treatment groups and the parasitemia level (p-value=0.7736). In contrast, a significant interaction was found between time and parasitemia level (p-value=0.0006).

Unlike the SOC population, two of the seven participants treated with imatinib alone experienced a rise in parasitemia and therefore had to be transferred to SOC (see representative time course in FIG. 1B). In contrast, the other patients in the imatinib monotherapy cohort experienced either an irregular or steady decline in parasitemia (FIG. 1C). In concordance with in vitro experiments showing that imatinib's efficacy depends on the stage of the parasite's life cycle during which it is first administered (i.e., owing to the fact that it primarily blocks parasite egress at the end of the parasite's life cycle (Kesely et al., PLOS One 11 (10: e0164895 (2016); Pantaleo et al., Blood [Internet] 130 (8): 1031 LP-1040 (Aug. 24, 2017); Kesely et al., PLOS One [Internet] 15 (11): e0242372 (Nov. 12, 2020)), the time-dependent efficacy of the imatinib monotherapy was anticipated and could be speculated to derive from the stage of the parasite's life cycle when treatment was initiated. Finally, even though a mixed ANOVA showed that the apparent accelerated decrease in pyrexia in the imatinib-treated cohort (FIG. 1D) did not achieve statistical significance (p-value=0.7029), we nevertheless elected to include the rate of pyrexia reduction as a secondary endpoint in the triple combination trial to determine whether it might be accelerated by imatinib.

Encouraged that imatinib might have anti-malarial activity, we next decided to compare the safety and efficacy of SOC alone (40 mg/day dihydroartemisinin+320 mg/day piperaquine) with the safety and efficacy of Im+SOC (i.e., 400 mg/day imatinib+40 mg/day dihydroartemisinin+320 mg/day piperaquine). For this purpose, 41 adult male participants with uncomplicated malaria were recruited from the same region of Vietnam and randomized into one of two cohorts that were treated for three consecutive days with either SOC or Im+SOC (Table 2). Evaluation of all adverse events revealed that the triple combination therapy was as safe as SOC, displaying no adverse events attributable to the added imatinib. Therefore, the primary endpoint of safety and tolerability was met for the Im+SOC triple combination therapy.

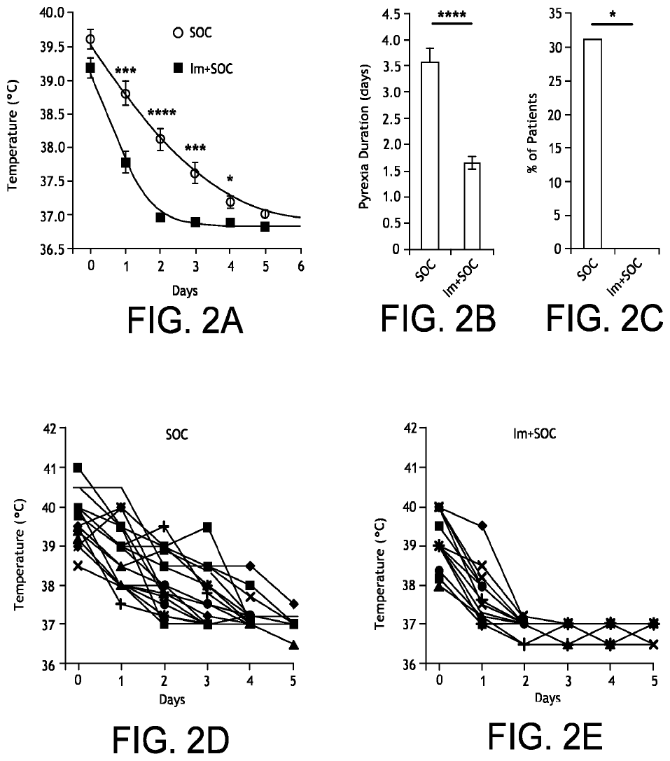

Although both cohorts entered the trial with similar levels of pyrexia (39.6±0.1° C. vs 39.2±0.2° C. for SOC vs Im+SOC; p=0.0501, NS; FIG. 2A), analysis of patient temperatures before, during and after the treatments demonstrated that body temperatures (i.e. a good measure of how a patient feels) returned to normal ˜2 days faster (1.65±0.12 versus 3.57±0.26 days; p=0.00002) in the triple combination than SOC therapy (FIG. 2B). Moreover, while all participants treated with Im+SOC (FIGS. 2C and 2E) experienced a monotonous decline in body temperature, >30% of participants in the SOC cohort (FIGS. 2C and 2D) experienced a second increase in temperature during at least one day of their therapy or follow-up period (p=0.0311). Consistent with these results, attending physicians noted that participants often reported feeling better sooner in the triple combination cohort than SOC cohort. Taken together, these data demonstrate that the secondary endpoint of faster resolution of pyrexia was met for the Im+SOC treatment cohort.

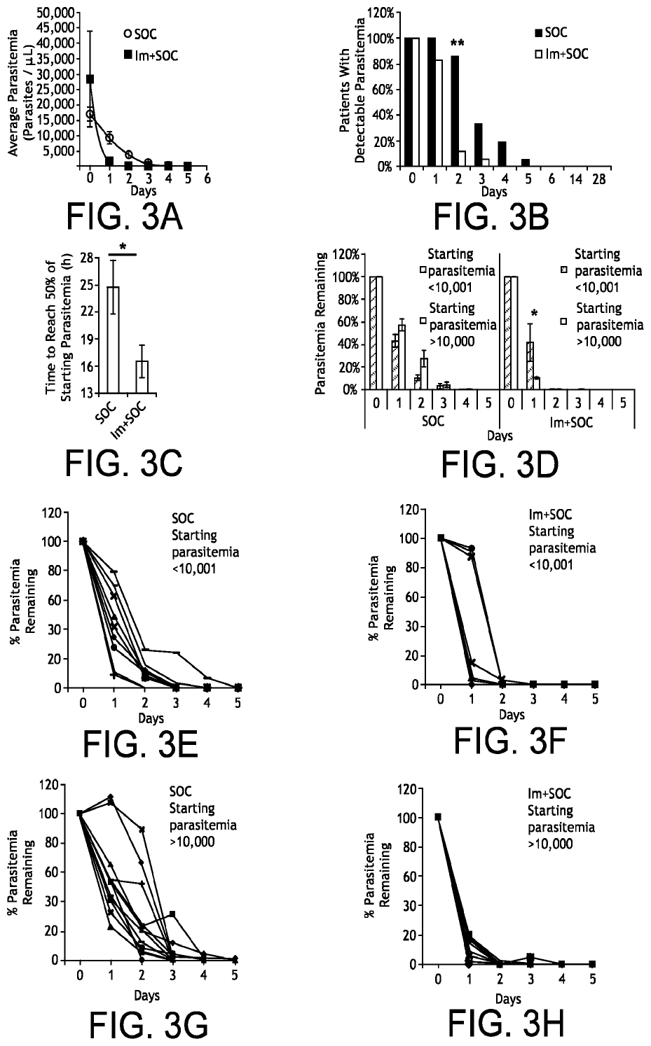

To assess whether the accelerated decline in body temperature might correlate with a more rapid elimination of parasitemia, we next compared the number of parasites/μL of peripheral blood in the two treatment groups. Although the average initial parasite concentration in peripheral blood did not differ significantly between SOC and Im+SOC cohorts (16,601±2099 parasites/μL versus 28,347±15,467 parasites/μL, respectively; p=0.2033), parasitemia decreased more rapidly in the triple combination than SOC cohort (FIG. 3A, panels A, B, C, D). Thus, 18% of participants receiving Im+SOC displayed no residual parasitemia at 24 hours post-ingestion of the initial dose of therapy (i.e., before receipt of their second dose), whereas none of the participants on SOC displayed an absence of parasitemia at the same 24-hours timepoint (FIG. 4B). Moreover, 88% of participants in the Im+SOC cohort were devoid of blood parasites by 48 hours post-initiation of therapy, with most of the remainder becoming parasite-free by day three. In contrast, only 14% of participants in SOC group were devoid of parasitemia at the end of day two and one third still retained measurable parasitemia after the full three-day course of therapy (FIG. 3B); i.e., confirming the established delayed parasite clearance in this region of Vietnam. (5) Therefore, the secondary endpoint of faster parasite clearance by the Im+SOC cohort was met.

Upon further scrutiny of the individual patient data in FIG. 3, it was noted that responses to the triple combination therapy were bimodal, with individuals initially diagnosed with high levels of parasitemia (panel H) responding more rapidly than patients initially diagnosed with low parasitemia (panel F). Indeed, all 10 patients presenting with >10,000 parasites/u L blood in the Im+SOC treatment group experienced a rapid decline in parasitemia (>80% in 24 hours; FIG. 3H). In contrast, patients in the SOC treatment cohort showed no such trend, with patients presenting at diagnosis with either high or low parasitemia responding similarly (FIG. 3, panels D, E, G). Importantly, no participant in the triple combination treatment group experienced recrudescence following completion of the three-day therapy (data collected for 42 days post therapy initiation).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as claimed herein.

The following embodiments are provided, the numbering of which is not to be construed as designating levels of importance:

1. A method of treating severe malaria in a subject, which method comprises administering to the subject an isoform of artemisinin, a hydrophobic amine, and a spleen tyrosine kinase (Syk) inhibitor in amounts effective to eliminate parasitemia within about 72 hours, whereupon the subject is treated for severe malaria.

2. The method of Embodiment 1, wherein the isoform of artemisinin is selected from the group consisting of artemisinin, dihydroartemisinin, artesunate, and artemether.

3. The method of claim 1 or 2, wherein the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

4. The method of Embodiment 1, 2 or 3, wherein the Syk inhibitor competes with adenosine triphosphate (ATP) for binding to Syk.

5. The method of Embodiment 4, wherein the Syk inhibitor comprises a bisarylanilino core that interacts with the gatekeeper amino acid residue Thr315 of the ATP binding pocket of BCR-ABL through hydrogen bond and Van der Waals interactions.

6. The method of Embodiment 4, wherein the Syk inhibitor is selected from the group consisting of imatinib, imatinib mesylate, and nilotinib.

7. The method of Embodiment 1, 2, or 3, wherein the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

8. The method of Embodiment 1, which method comprises administering to the subject about 40 mg/day dihydroartemisinin, about 320 mg/day piperaquine, and about 400 mg/day imatinib.

9. A method of treating uncomplicated malaria in a subject, which method comprises administering to the subject an isoform of artemisinin, a hydrophobic amine, and a spleen tyrosine kinase (Syk) inhibitor in amounts effective to eliminate parasitemia within about 72 hours, wherein, when the isoform of artemisinin is dihydroartemisinin and the hydrophobic amine is piperaquine, the Syk inhibitor is other than imatinib or imatinib mesylate, whereupon the subject is treated for uncomplicated malaria.

10. The method of Embodiment 9, wherein the isoform of artemisinin is selected from the group consisting of artemisinin, dihydroartemisinin, artesunate, and artemether.

11. The method of Embodiment 9 or 10, wherein the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

12. The method of Embodiment 9, 10 or 11, wherein the Syk inhibitor competes with adenosine triphosphate (ATP) for binding to Syk.

13. The method of Embodiment 12, wherein the Syk inhibitor comprises a bisarylanilino core that interacts with the gatekeeper amino acid residue Thr315 of the ATP binding pocket of BCR-ABL through hydrogen bond and Van der Waals interactions.

14. The method of Embodiment 12, wherein the Syk inhibitor is nilotinib.

15. The method of Embodiment 9, 10 or 11, wherein the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

16. A method of treating uncomplicated malaria in a subject, which method comprises administering to the subject about 40 mg/day dihydroartemisinin, about 320 mg/day piperaquine, and about 400 mg/day imatinib, whereupon the subject is treated for uncomplicated malaria.

17. A unitary, oral dosage form comprising an isoform of artemisinin, a hydrophobic amine, and a spleen tyrosine kinase (Syk) inhibitor in amounts effective to treat parasitemia.

18. The unitary, oral dosage form of Embodiment 17, wherein the isoform of artemisinin is selected from the group consisting of artemisinin, dihydroartemisinin, artesunate, and artemether.

19. The unitary, oral dosage form of Embodiment 17 or 18, wherein the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

20. The unitary, oral dosage form of Embodiment 17, 18 or 19, wherein the Syk inhibitor competes with adenosine triphosphate (ATP) for binding to Syk.

21. The unitary, oral dosage form of Embodiment 20, wherein the Syk inhibitor comprises a bisarylanilino core that interacts with the gatekeeper amino acid residue Thr315 of the ATP binding pocket of BCR-ABL through hydrogen bond and Van der Waals interactions.

22. The unitary, oral dosage form of Embodiment 20, wherein the Syk inhibitor is selected from the group consisting of imatinib, imatinib mesylate, and nilotinib.

23. The unitary, oral dosage form of Embodiment 17, 18 or 19, wherein the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

24. The unitary, oral dosage form of Embodiment 17 comprising dihydroartemisinin, piperaquine, and imatinib.

25. The unitary, oral dosage form of Embodiment 24 comprising about 40 mg dihydroartemisinin, about 320 mg piperaquine, and about 400 mg imatinib.

26. The unitary, oral dosage form of Embodiment 17 comprising an isoform of artemisinin, a hydrophobic amine, and a Syk inhibitor other than imatinib or imatinib mesylate.

27. The unitary, oral dosage form of Embodiment 26, wherein the isoform of artemisinin is selected from the group consisting of artemisinin, dihydroartemisinin, artesunate, and artemether.

28. The unitary, oral dosage form of Embodiment 26 or 27, wherein the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

29. The unitary, oral dosage form of Embodiment 26, 27 or 28, wherein the Syk inhibitor competes with ATP for binding to Syk.

30. The unitary, oral dosage form of Embodiment 29, wherein the Syk inhibitor comprises a bisarylanilino core that interacts with the gatekeeper amino acid residue Thr315 of the ATP binding pocket of BCR-ABL through hydrogen bond and Van der Waals interactions.

31. The unitary, oral dosage form of Embodiment 29, wherein the Syk inhibitor is nilotinib.

32. The unitary, oral dosage form of Embodiment 26, 27 or 28, wherein the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

33. The unitary, oral dosage form of Embodiment 26 comprising dihydroartemisinin, piperaquine, and a Syk inhibitor other than imatinib or imatinib mesylate.

34. The unitary, oral dosage form of Embodiment 33 comprising about 40 mg dihydroartemisinin, about 320 mg piperaquine, and about 400 mg of a Syk inhibitor other than imatinib or imatinib mesylate.

35. A kit comprising multiple unitary, oral dosage forms of any one of Embodiments 17-25 and instructions for administration for severe malaria.

36. The kit of Embodiment 35, which comprises two or three unitary, oral dosage forms.

37. A kit comprising multiple unitary, oral dosage forms of any one of Embodiments 26-34 and instructions for administration for uncomplicated malaria.

38. The kit of Embodiment 37, which comprises at least two or three unitary, oral dosage forms.

1. A method of treating severe malaria in a subject, which method comprises administering to the subject an isoform of artemisinin, a hydrophobic amine, and a spleen tyrosine kinase (Syk) inhibitor in amounts effective to eliminate parasitemia within about 72 hours, whereupon the subject is treated for severe malaria;
wherein:
a subject suffering from pyrexia associated with the malaria experiences a monotonous decline in body temperature and does not experience a second increase in temperature during at least one day of therapy.

2. The method of claim 1, wherein the isoform of artemisinin is selected from the group consisting of artemisinin, dihydroartemisinin, artesunate, and artemether; and/or
the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

3. (canceled)

4. The method of claim 1, wherein:
the Syk inhibitor competes with adenosine triphosphate (ATP) for binding to Syk;
the Syk inhibitor comprises a bisarylanilino core that interacts with the gatekeeper amino acid residue Thr315 of the ATP binding pocket of BCR-ABL through hydrogen bond and Van der Waals interactions;
the Syk inhibitor is selected from the group consisting of imatinib, imatinib mesylate, and nilotinib; or
the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

5. (canceled)

6. (canceled)

7. (canceled)

8. (canceled)

9. A method of treating uncomplicated malaria in a subject, which method comprises administering to the subject an isoform of artemisinin, a hydrophobic amine, and a spleen tyrosine kinase (Syk) inhibitor in amounts effective to eliminate parasitemia within about 72 hours, wherein, when the isoform of artemisinin is dihydroartemisinin and the hydrophobic amine is piperaquine, the Syk inhibitor is other than imatinib or imatinib mesylate, whereupon the subject is treated for uncomplicated malaria;
wherein:
a subject suffering from pyrexia associated with the malaria experiences a monotonous decline in body temperature and does not experience a second increase in temperature during at least one day of therapy.

10. The method of claim 9, wherein;
the isoform of artemisinin is selected from the group consisting of artemisinin, dihydroartemisinin, artesunate, and artemether; and/or
the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

11. (canceled)

12. The method of claim 9, wherein:
the Syk inhibitor competes with adenosine triphosphate (ATP) for binding to Syk;
the Syk inhibitor comprises a bisarylanilino core that interacts with the gatekeeper amino acid residue Thr315 of the ATP binding pocket of BCR-ABL through hydrogen bond and Van der Waals interactions;
the Syk inhibitor is nilotinib; or
the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

13. (canceled)

14. (canceled)

15. (canceled)

16. A method of treating uncomplicated malaria in a subject, which method comprises administering to the subject about 40 mg/day dihydroartemisinin, about 320 mg/day piperaquine, and about 400 mg/day imatinib, whereupon the subject is treated for uncomplicated malaria.

17. A unitary, oral dosage form comprising an isoform of artemisinin, a hydrophobic amine, and a spleen tyrosine kinase (Syk) inhibitor in amounts effective to treat parasitemia.

18. The unitary, oral dosage form of claim 17, wherein the isoform of artemisinin is selected from the group consisting of artemisinin, dihydroartemisinin, artesunate, and artemether; and/or
the hydrophobic amine is selected from the group consisting of lumefantrine, mefloquine, amodiaquine, the combination of sulfadoxine and pyrimethamine, piperaquine, chloroquine, and the combination of chlorproguanil and dapsone.

19. (canceled)

20. The unitary, oral dosage form of claim 17, wherein:
the Syk inhibitor competes with adenosine triphosphate (ATP) for binding to Syk;
the Syk inhibitor comprises a bisarylanilino core that interacts with the gatekeeper amino acid residue Thr315 of the ATP binding pocket of BCR-ABL through hydrogen bond and Van der Waals interactions;
the Syk inhibitor is selected from the group consisting of imatinib, imatinib mesylate, and nilotinib;
the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

21. (canceled)

22. (canceled)

23. (canceled)

24. The unitary, oral dosage form of claim 17 comprising dihydroartemisinin, piperaquine, and imatinib.

25. (canceled)

26. The unitary, oral dosage form of claim 17 comprising an isoform of artemisinin, a hydrophobic amine, and a Syk inhibitor other than imatinib or imatinib mesylate.

27. The unitary, oral dosage form of claim 26, wherein:
the isoform of artemisinin is selected from the group consisting of artemisinin, dihydroartemisinin, artesunate, and artemether; and/or
the hydrophobic amine is selected from the group consisting of lumefantrine, mefloquine, amodiaquine, the combination of sulfadoxine and pyrimethamine, piperaquine, chloroquine, and the combination of chlorproguanil and dapsone;

28. (canceled)

29. The unitary, oral dosage form of claim 26, wherein:
the Syk inhibitor competes with ATP for binding to Syk;
the Syk inhibitor comprises a bisarylanilino core that interacts with the gatekeeper amino acid residue Thr315 of the ATP binding pocket of BCR-ABL through hydrogen bond and Van der Waals interactions;
the Syk inhibitor is nilotinib; or
the Syk inhibitor is Syk inhibitor II, Syk inhibitor IV, R406, R788, P505-15, 3,4-methylenedioxy-β-nitrostyrene, R112, GS-9973, piceatannol, dasatinib, bosutinib, or ponatinib.

30. (canceled)

31. (canceled)

32. (canceled)

33. The unitary, oral dosage form of claim 26 comprising dihydroartemisinin, piperaquine, and a Syk inhibitor other than imatinib or imatinib mesylate.

34. (canceled)

35. A kit comprising multiple unitary, oral dosage forms of claim 17 and instructions for administration for severe malaria.

36. (canceled)

37. A kit comprising multiple unitary, oral dosage forms of claim 26 and instructions for administration for uncomplicated malaria.

38. (canceled)

39. The method of claim 1, wherein:
the pyrexia is resolved faster relative to a standard-of-care (SOC) cohort;
the pyrexia is resolved in less than about two days;
parasitemia is more rapidly eliminated relative to a SOC cohort;
the subject experiences a greater than 80% reduction in the number of parasites per microliter of blood in about 24 hours relative to a SOC cohort; or
parasitemia is eliminated in about 24 hours to about 48 hours post-ingestion after an initial dose of therapy.

40. (canceled)

41. (canceled)

42. (canceled)

43. (canceled)

44. The method of claim 1, wherein;
a response to therapy is bimodal; or
the subject experiences no recrudescence following completion of a three-day therapy.

45. (canceled)

46. The method of claim 9, wherein:
the pyrexia is resolved faster relative to a standard-of-care (SOC) cohort;
the pyrexia is resolved in less than about two days;
parasitemia is more rapidly eliminated relative to a SOC cohort;
the subject experiences a greater than 80% reduction in the number of parasites per microliter of blood in about 24 hours relative to a SOC cohort;
parasitemia is eliminated in about 24 hours to about 48 hours post-ingestion after an initial dose of therapy;
a response to therapy is bimodal; or
the subject experiences no recrudescence following completion of a three-day therapy.

47. (canceled)

48. (canceled)

49. (canceled)

50. (canceled)

51. (canceled)

52. (canceled)
